(19) United States
(12) Patent Application Publication
(10) Pub. No.: US 2011/0251616 A1
(43) Pub. Date: Oct. 13, 2011

(54) EXPANDABLE REAMER AND METHOD OF USE

(75) Inventors: Said Osman, Russellville, AL (US); Scott Jones, McMurray, PA (US); Chris Straight, Leesburg, VA (US)

(73) Assignee: K2M, Inc., Leesburg, VA (US)

(21) Appl. No.: 13/084,904

(22) Filed: Apr. 12, 2011

Related U.S. Application Data
(60) Provisional application No. 61/322,947, filed on Apr. 12, 2010.

(51) Int. Cl. A61B 17/16; A61F 2/44

(52) U.S. Cl. 606/80; 623/17.16

(57) ABSTRACT

A surgical reamer assembly includes a dilator having a longitudinal passage extending therethrough for receiving a guidewire and a tubular channel positioned over the dilator. The surgical reamer assembly also includes an expandable reamer head coupled to an elongated shaft, such that the expandable reamer head is insertable through the tubular channel once the dilator and guidewire are removed.

### CROSS REFERENCE TO RELATED APPLICATIONS

The present application claims priority from U.S. Provisional Patent Application No. 61/322,947 filed Apr. 12, 2010, the contents of which are incorporated herein by reference.

### BACKGROUND

1. Technical Field

The present disclosure relates to a surgical reamer and, more particularly, to a method for treating spinal conditions by removing disc material between vertebral bodies via the surgical reamer.

2. Background of Related Art

The human spine includes thirty-three vertebrae. The vertebrae interconnect with one another to form a spinal column. Each vertebrae has a cylindrical bony body (vertebral body), two pedicles extending from the vertebral body, a lamina extending from the pedicles, two wing-like projections extending from the pedicles, a spinous process extending from the lamina, a pars interarticularis, two superior facets extending from the pedicles, and two inferior facets extending from the lamina. The vertebrae are separated and cushioned by thin pads of tough, resilient fiber known as inter-vertebral discs (or discs). Inter-vertebral discs provide flexibility to the spine and act as shock absorbers during activity. A small opening (foramen) located between each vertebra allows passage of nerves. When the vertebrae are properly aligned, the nerves pass through without a problem. However, when the vertebrae are misaligned or a constriction is formed in the spinal canal, the nerves get compressed and may cause back pain, leg pain, or other neurological disorders.

Disorders of the spine that may cause misalignment of the vertebrae or constriction of the spinal canal include spinal injuries, infections, tumor formation, herniation of the inter-vertebral discs (i.e., slippage or protrusion), arthritic disorders, and scoliosis. In these pathological circumstances, surgery may be tried to either decompress the neutral elements and/or fuse adjacent vertebral segments. Decompression may involve laminectomy, discectomy, or corpectomy. Laminectomy involves the removal of inter-vertebral discs. Corpectomy involves the removal of the vertebral body, as well as the adjacent intervertebral discs.

A number of spinal surgical devices may be used to remove the disc material between the vertebral bodies to aid in decompressing the nerve roots and/or commencing a discectomy procedure. It is preferred that these procedures minimize the disruption to the surrounding tissue and vasculature so as to promote a more rapid healing process. Therefore, a need exists for a minimally invasive method for removing spinal disc material.

### SUMMARY

A surgical reamer assembly includes a dilator having a longitudinal passage extending therethrough for receiving a guidewire and a tubular channel positioned over the dilator. The surgical reamer assembly also includes an expandable reamer head coupled to an elongated shaft, such that the expandable reamer head is insertable through the tubular channel once the dilator and guidewire are removed.

A proximal end of the shaft is coupled to a driving source configured to rotatably actuate the expandable reamer head. Additionally, the expandable reamer head is configured to be shaped as one of conical, spherical and cylindrical.

In another exemplary embodiment, the tubular channel includes an irrigation port for (i) communicating a fluid such as sterile saline or another biocompatible fluid to enter the tubular channel and (ii) moving loose disc material away from the expandable reamer head. The tubular channel has a threaded distal end configured to be fixedly secured to endplates of one or more vertebral bodies. Moreover, the tubular channel is configured to be releasably secured to a handle portion such that a substantially “L-shape” configuration is formed.

In yet another exemplary embodiment, the expandable reamer includes at least one cutting element. The at least one cutting element may be a pair of opposed cutting blades. Additionally, in an expanded state the pair of opposed cutting blades are exposed and in a retracted state the pair of opposed cutting blades are concealed within the expandable reamer head.

A method of removing disc material between vertebral bodies is presented. The method includes the steps of introducing a dilator having a guidewire extending therethrough into a disc space for separating muscle and tissue and positioning a tube channel over the dilator, and removing the dilator and the guidewire. The method further includes the steps of inserting an expandable reamer head through the tube channel for accessing the disc space, expanding at least one portion of the expandable reamer head to expose at least one cutting element, and manipulating the expandable reamer head to remove the disc material positioned about the disc space.

The method may also include a plurality of reaming assemblies, each including an expandable reamer head.

One reaming assembly of the plurality of reaming assemblies is placed on an ipsilateral side of a disc space and another reaming assembly of the plurality of reaming assemblies is placed on a contralateral side of the disc space.

Features from any of the above-mentioned embodiments may be used in combination with one another in accordance with the present disclosure. Other features and advantages of the present disclosure will become apparent to those of ordinary skill in the art through consideration of the ensuing description, the accompanying drawings, and the appended claims.

### BRIEF DESCRIPTION OF THE DRAWINGS

Embodiments of the presently disclosed expandable reamer assembly are described herein with reference to the accompanying drawings, wherein:

FIG. 1 is a perspective view of a dilator, in accordance with the present disclosure;

FIG. 2 is a perspective view of a tubular channel, in accordance with the present disclosure;

FIG. 3A is a perspective view of a reamer in a closed or retracted position, in accordance with the present disclosure;

FIG. 3B is a magnified view of a distal end of the reamer of FIG. 3A in a closed position, in accordance with the present disclosure;

FIG. 3C is a perspective view of a reamer in an open or expanded position, in accordance with the present disclosure;

FIG. 3D is a magnified view of a distal end of the reamer of FIG. 3C in an open position, in accordance with the present disclosure;

FIG. 4A is a perspective view of the reamer of FIG. 3A partially inserted through the tubular channel of FIG. 2, in accordance with the present disclosure;

FIG. 4B is a perspective view of the reamer of FIG. 3A fully inserted through the tubular channel of FIG. 2, and fully locked into place, in accordance with the present disclosure;

FIG. 4C is a perspective view of the reamer of FIG. 3C expanded at distal end after being fully inserted through the tubular channel of FIG. 2, in accordance with the present disclosure;

FIG. 5 is a perspective view of the expandable reamer assembly, where a fluid source is connected to the expandable reamer assembly, in accordance with the present disclosure; and

FIG. 6 is a perspective view of a plurality of expandable reamer assemblies working to remove disc material between vertebral discs of the spine, the plurality of reamer assemblies entering the disc space postero-laterally, in accordance with the present disclosure.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplification set out herein illustrates embodiments of the present disclosure, in several forms, and such exemplifications are not to be construed as limiting the scope of the present disclosure in any manner.

### DETAILED DESCRIPTION OF THE EMBODIMENTS

Embodiments of the presently disclosed reamer assembly will now be described in detail with reference to the drawing figures wherein like reference numerals identify similar or identical elements. In the drawings and in the description which follows, the term “proximal”, as is traditional, will refer to the end of the reamer assembly which is closest to the operator while the term “distal” will refer to the end of the reamer assembly which is farthest from the operator.

The exemplary embodiments of the present disclosure provide for an improved disc preparation method and reamer assembly. One skilled in the art will recognize that the present disclosure is not limited to the use in the field of spine surgery, and that the instruments and methods presented herein may be adapted for use with any suitable surgical device or apparatus. Those skilled in the art will appreciate that the present disclosure may be implemented in a number of different applications and embodiments and is not specifically limited in its application to the particular example embodiments illustrated herein.

The exemplary embodiments of the present disclosure relate to an expandable reamer utilized in association with a dilator and tubular channel in a minimally invasive manner to remove the disc material between the vertebral bodies and to roughen the endplates to reduce bleeding and to promote a proper fusion with the intervertebral implant of choice.

In another exemplary embodiment, a method where a guidewire is placed into a disc space followed by a dilator to gently separate the muscle and tissue approaching the disc is presented. A tubular channel is inserted over the dilator, and the dilator and guidewire are removed. The tubular channel is utilized by the expandable reamer for access to the disc space. Once the reamer is inserted through the tubular channel, it is expanded such that the cutting elements of the reamer are exposed. The reamer is now rotated back and forth to abrade the disc material and clean and/or roughen the endplates. Of course, the reamer may also be moved axially to abrade the disc material and/or clean/roughen the endplates. Therefore, the expandable reamer head is axially and rotatably movable.

In yet another exemplary embodiment, the tubular channel includes an irrigation port for allowing sterile saline or another suitable fluid to enter the tubular channel and aid in cleaning the cutting surfaces of the reamer and moving the loose disc material away from the reamer so as to not collect on the cutting surfaces of the reamer. The tubular channel also includes a threaded distal end that may be fixed to the endplates so as to minimize any repositioning of the tubular channel.

In yet another exemplary embodiment, there are two dilators, two tubular channels, and two reamers so as to form a plurality of reamer assemblies, such that one reamer assembly is placed on the contralateral side and another reamer assembly is placed on the ipsilateral side of the disc space. One reamer assembly may be used for suctioning of the disc material, as well as for providing additional reaming capability for removing the disc material. The reamer assemblies may also be used as ports for an endoscope so that viewing of the disc space is possible to ensure complete discectomy and adequate endplate preparation.

Various embodiments of the intervertebral disc reamer assembly will be described with reference to the drawings, where like elements are represented by like reference numerals.

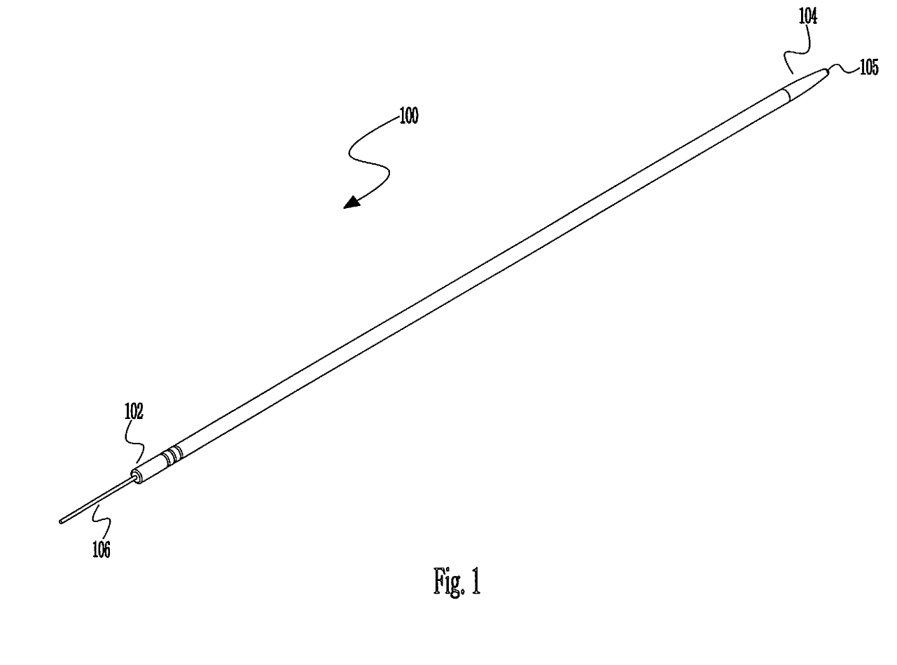

Referring to FIG. 1, a perspective view of a dilator 100, in accordance with the present disclosure is presented.

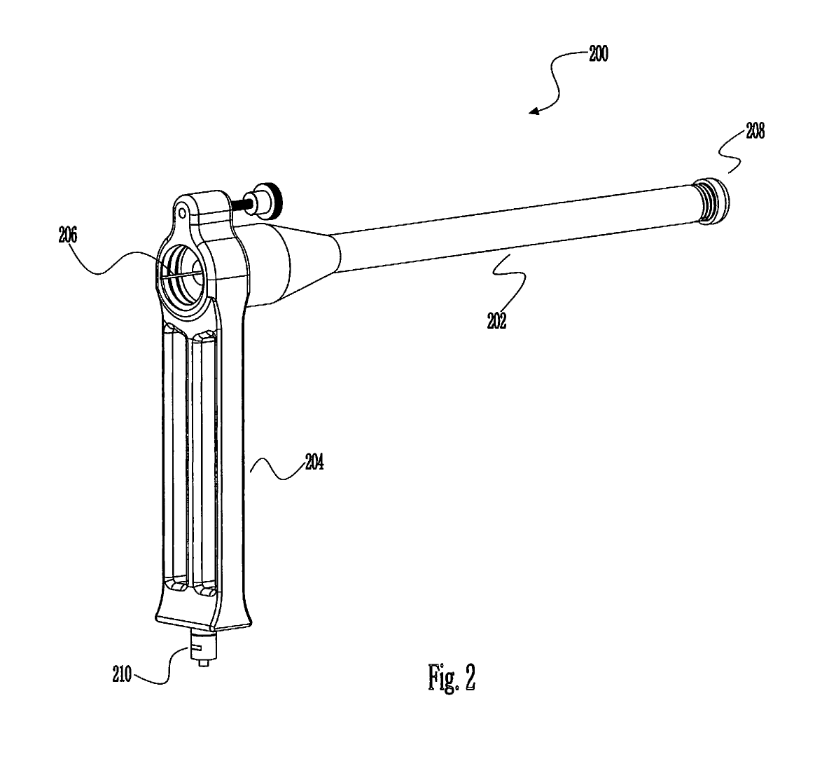

The dilator 100 is configured and dimensioned to be received through a tubular channel 200 (see FIG. 2), the dilator 100 having a distal end tip 104. Dilator 100 includes a longitudinal passage therethrough having an opening 102 for receiving a guidewire 106 therethrough. Additionally, an opening 105 may be located at a distal end of the dilator 100 for receiving the guidewire 106 extending through the longitudinal passage of the dilator 100.

Referring to FIG. 2, a perspective view of a tubular channel 200, in accordance with the present disclosure is presented.

The tubular channel 200 includes a shaft portion 202 and a handle portion 204. An opening 206 is proximally located for receiving the dilator 100 of FIG. 1. Once the dilator 100 is inserted through the tubular channel 200, the tip 104 of the dilator 100 exits the distal end 208 of the shaft portion 202. It is also contemplated that a fluid connector 210 may be positioned at a bottom end of the handle portion 204. The fluid connector 210 may be used to connect a fluid source 510, as described below with reference to FIG. 5.

The tubular channel 200 is configured to be releasably secured to the handle portion 204 such that a substantially “L-shaped” configuration is formed.

In an exemplary embodiment, the tubular member 200 may be made from stainless steel. In alternative embodiments, the tubular member 200 may be made from any other suitable materials.

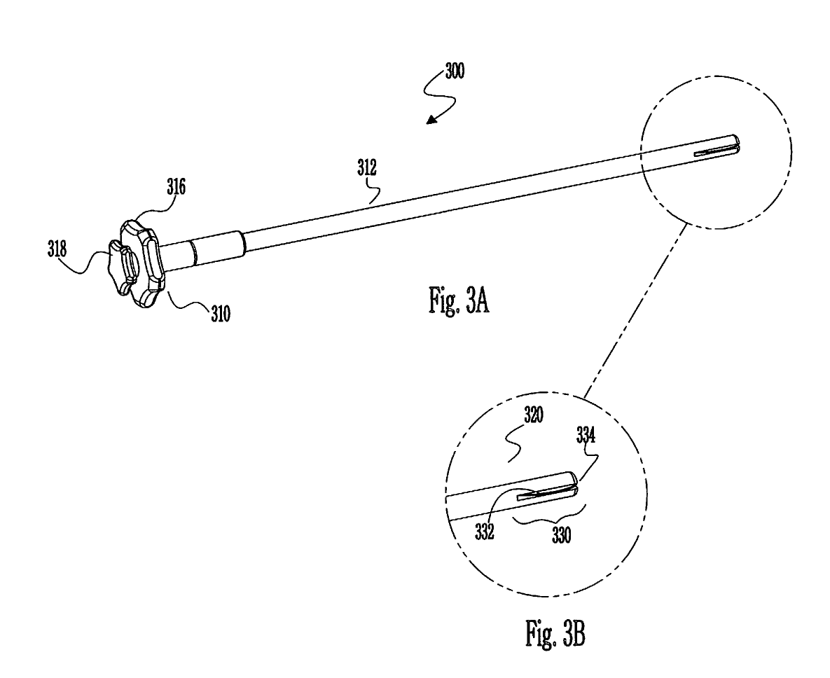

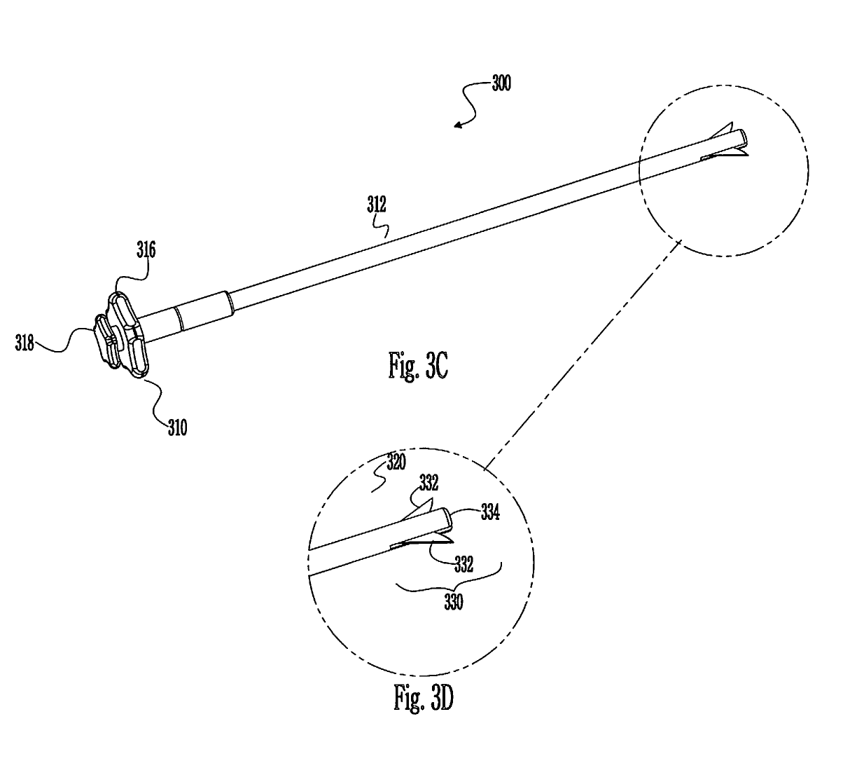

Referring to FIGS. 3A-3D, a reamer 300 is presented, where the distal end of the reamer 300 is expanded and retracted between first and second positions.

Reamer 300 includes an actuation mechanism 310 at the proximal end of the shaft 312. The actuation mechanism 310 may include a first knob 316 for rotating the reamer 300 and a second knob 318 for controlling blade retraction/expansion. Thus, the proximal end of the shaft 312 may be coupled to a driving source (e.g., first knob 316 and second knob 318) configured to rotatably and axially actuate the expandable reamer head portion 330. At the distal end 320 of the reamer 300 is a reamer head portion 330. The reamer head portion 330 includes blades 332 and a head member 334.

In the retracted position, as shown in FIG. 3B, the blades 332 are contracted within the distal end of the shaft 312 of the reamer 300. The blades 332 may also be referred to herein as cutting elements 332. The cutting elements 332 may be a pair of opposed cutting blades.

The reamer 300 may be formed using different materials, including various metal alloys, plastic materials and the like. However, one skilled in the art may contemplate using any other suitable materials.

The diameter of the shaft 312 is sized such that the distal end of the shaft 312 may be inserted into a patient's body with the distal end 320 placed against a diseased disc or bone without the shaft 312 having undue interference with other anatomical organs. The shaft 312 is configured to transmit torque from a suitable rotary power source (e.g., first knob 316 of actuation mechanism 310) from a proximal end to a distal end 320 of reamer 300, and the shaft 312 is configured to cooperate in expanding and retracting distal end portions of the reamer 300.

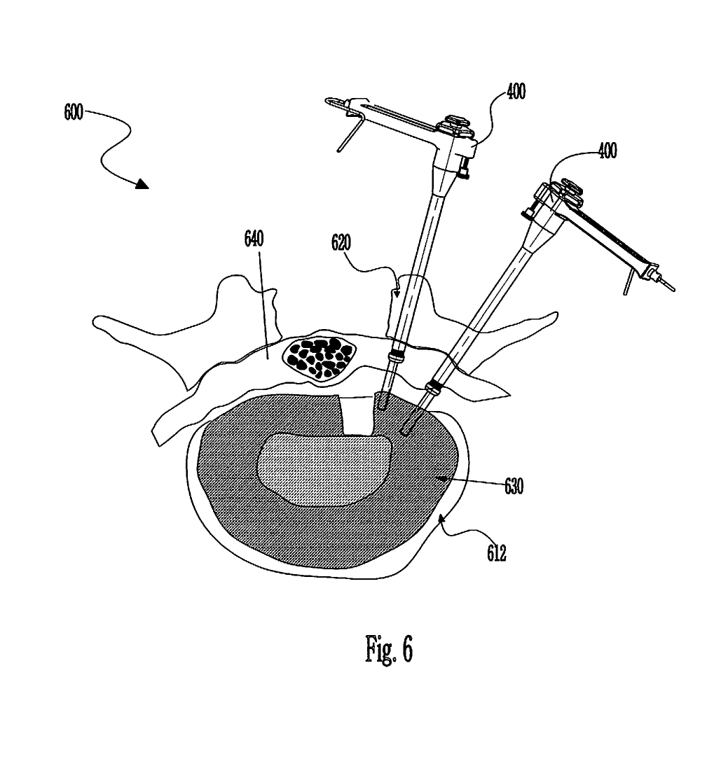

The expandable reamer head portion 330 is configured to be shaped as one of conical, spherical and cylindrical. For example, in FIGS. 3C-3D, the distal end 320 of the shaft 312 is shown in an expanded position, where the blades 332 extend away from the shaft 312 in order to come into contact with disc material 630 (see FIG. 6). Additionally, the head member 334 extends longitudinally outward to allow the blades 332 to be fully expanded, as seen in FIG. 3D.

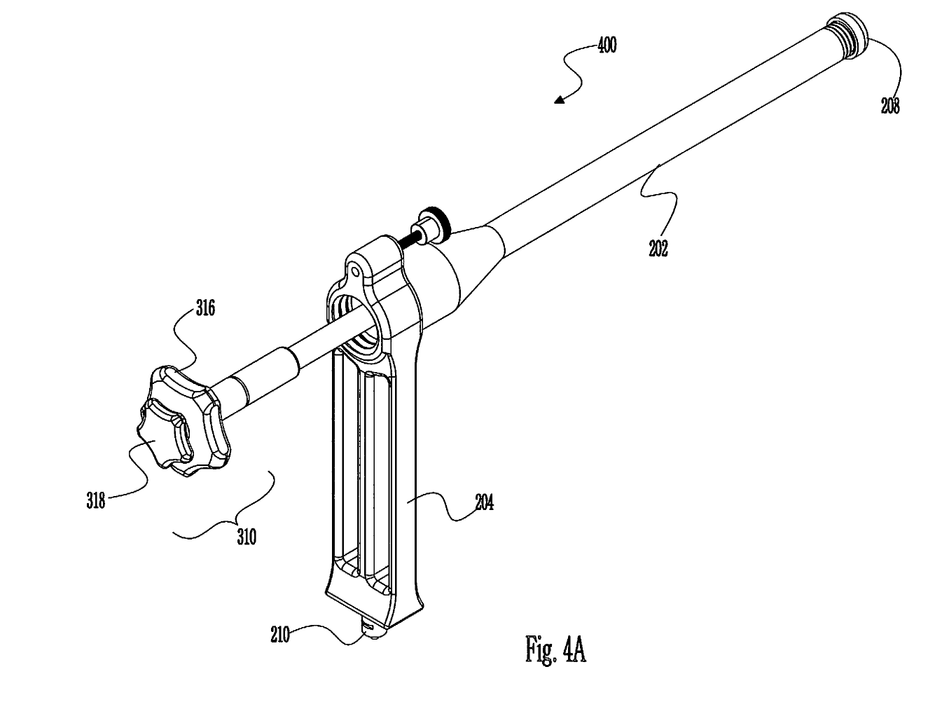

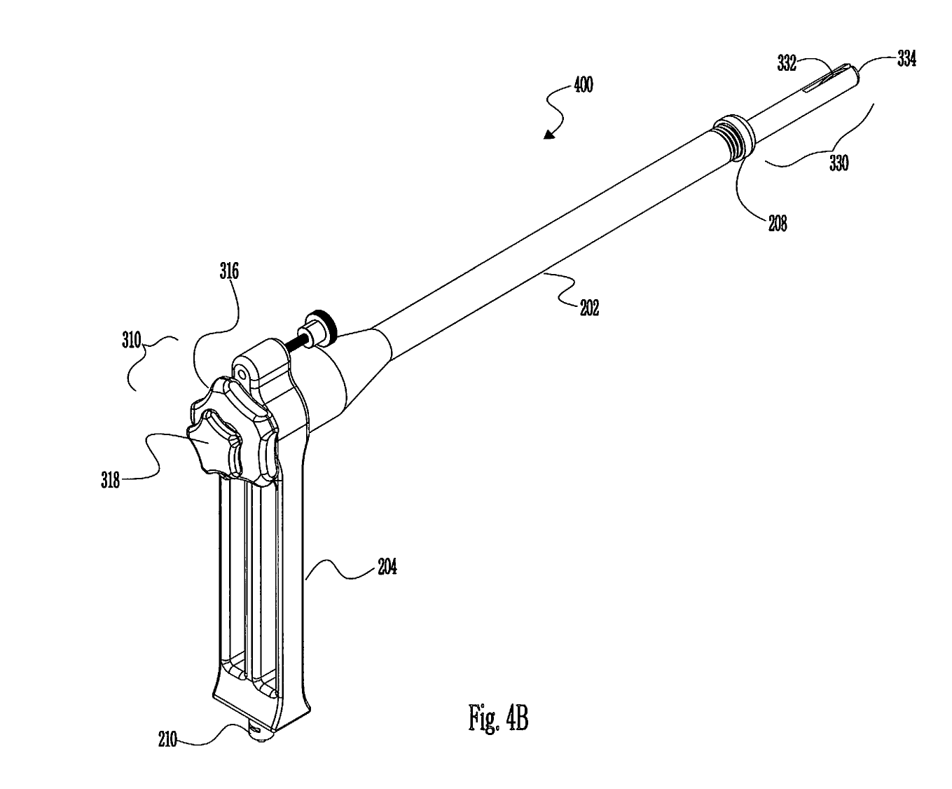

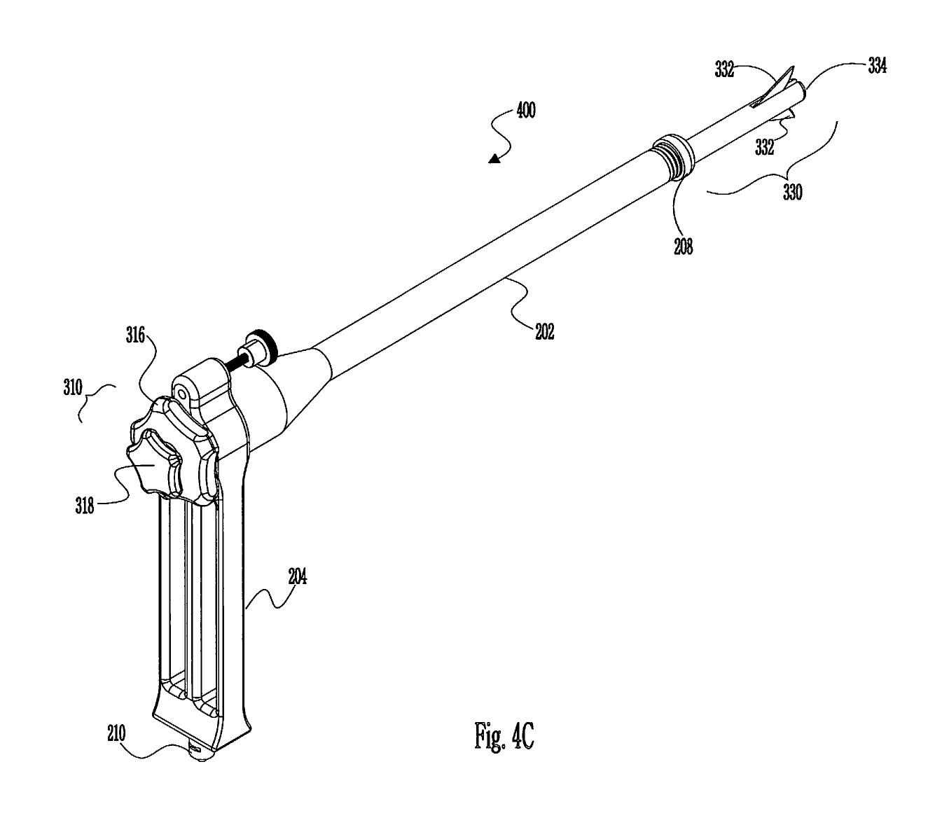

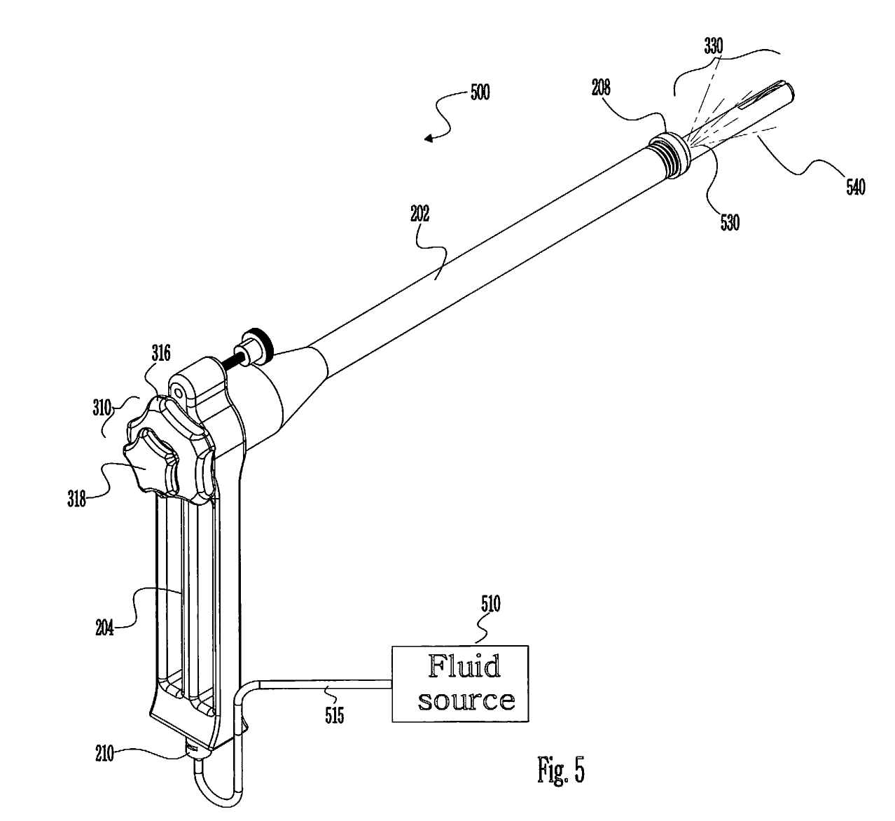

Referring to FIGS. 4A-4C, a reamer assembly 400 is presented, where the reamer 300 of FIG. 3A is inserted through the tubular member 200 of FIG. 2, such that the distal end of the reamer 300 is expanded and retracted between first and second positions.

FIG. 4A merely illustrates the reamer 300 of FIG. 3A being inserted into the tubular member 200 of FIG. 2.

FIG. 4B illustrates the reamer 300 fully inserted and locked into the tubular member 200. Additionally, the blades 332 are shown in a retracted (or non-exposed) position.

Therefore, the surgical reamer assembly 400 includes a dilator 100 having a longitudinal passage extending therethrough for receiving a guidewire 106 and a tubular channel 200 inserted over the dilator 100. The expandable reamer head portion 330 is coupled to the elongated shaft 312 such that the expandable reamer head portion 330 is inserted through the tubular channel 200 once the dilator 100 and guidewire 106 are removed.

In FIG. 4C, the blades 332 may be extended to an expanded position while rotating the reamer 300 via the actuation means 310 (e.g., a first knob 316), thereby forming a cavity in bone structure or removing disc material 630 between vertebral bodies (see FIG. 6). The blades 332 may return to their original position (contracted or non-exposed position) by the force applied by the structure being reamed as the reamer 300 is withdrawn from the cavity formed. In an alternative embodiment, the blades 332 may also be spring-loaded or otherwise biased to their original contracted position.

Thus, the reaming assembly 400 has expandable blades 332 that may be actuated from at or near the distal end of the reaming assembly 400 so that the surgeon may do so while the reamer head portion 330 is inside the patient. The expansion actuation may be done by a gear system (not shown) that transmits rotation to the first knob 316 or other control member of the reaming assembly 400 to rotate the elongated member 312 that is preferably coaxial with the central axis of the reaming assembly 400 and that extends down to the reamer head portion 330.

Preferably, multiple cutting blades 332 are provided, wherein at least one has a cutting edge extending greater than 180 degrees, or a group of cutting edges that together total greater than 180 degrees. Alternatively, a combination of two or more blades 332 may have cutting edges that, when the reamer head portion 330 is rotated 360 degrees, together are capable of cutting greater than 180 degrees. This way, no matter what the orientation of the reamer head portion 330 is in the vertebral body, the reamer head portion 330 may cut approximately 180 degrees. The expansion of the reamer head portion 330 is done with preferred structure and methods that provide extremely accurate reaming of various hemispherical diameters.

In an alternative embodiment, a movable blade 332 of the present disclosure may be removable and/or replaceable. Accordingly, such a configuration may allow for the expandable reamer head portion 330 of the present disclosure to be easily reconfigured for different diameters or repaired.

In another alternative embodiment, differently sized and/or spaced movable blades 332 may be configured so that (i) a first borehole diameter may be drilled at a first rate, and a second borehole diameter may be drilled at a second rate or (ii) different cutting edges are used to remove disc material 630 from a surgical site 600 (see FIG. 6).

Referring to FIG. 5, a perspective view of the reamer assembly 500, where a fluid source 510 is connected to the reamer assembly 500, in accordance with the present disclosure is presented.

The reamer assembly 500 includes a fluid source 510 for spraying liquid 540 via a nozzle 530. The nozzle 530 may be positioned at a distal end 208 of the shaft portion 202 of the tubular member 200. Of course, one skilled in the art may contemplate positioning the nozzle 530 and the fluid source 510 on or about any portion of the reamer assembly 500.

Thus, the tubular channel 200 includes a fluid source 510 for allowing, for example, sterile saline to enter the tubular channel 200 and travel a path 520 to aid in cleaning the cutting surfaces of the reamer assembly 500, as well as moving the loose disc material 630 (see FIG. 6) away from the reamer assembly 500 so as to not collect on the cutting surfaces of the reamer assembly 500. The tubular channel 200 also includes a threaded distal end 208 that may be fixed to the endplates (not shown) so as to minimize any repositioning of the tubular channel 200.

Referring to FIG. 6, a perspective view of a plurality of reamer assemblies 400 working to remove disc material 630 is presented, where the plurality of reamer assemblies 400 are positioned postero-laterally with respect to the disc space.

The surgical site 600 illustrates a top view of a vertebral body 612 such that disc material 630 is positioned on top of the vertebral body 612. A plurality of surgical reamer assemblies 400 may access the disc space in order to remove the disc material 630. The reamer assemblies 400 access the disc material 630 postero-laterally between the pedicle portion (not shown) and the spinous process portion 620. The spinal nerve 640 may be caused to be displaced by, for example, a forceps, in order to better position the reamer assemblies 400.

In FIG. 6, there are two dilators 100, two tubular channels 200, and two reamers 300 so as to form a plurality of reamer assemblies 400, such that one reamer assembly 400 is placed on the contralateral side and another reamer assembly 400 is placed on the ipsilateral side of the disc space. One reamer assembly 400 may be used for suctioning of the disc material 630, as well as for providing additional reaming capability for removing the disc material 630.

In an alternative embodiment, the reamer assemblies 400 may also be used as ports for an endoscope so that viewing of the disc space is possible to ensure complete discectomy and adequate endplate preparation.

Thus, in operation, the disc material 630 is removed from the surgical site 600 by introducing a dilator 100 having a guidewire 106 extending therethrough into the disc space for separating muscle and tissue. Then, in accordance with the method, the step of inserting a tube channel 200 over the dilator 100 is performed. The dilator 100 having the guidewire 106 extending therethrough is removed and the expandable reamer head portion 330 is inserted through the tube channel 200 for accessing the disc space. Then, the step of expanding at least one portion of the expandable reamer head 330 to expose at least one cutting element 332 is performed. Once the cutting elements 332 are exposed, the method performs the step of rotating the expandable reamer head portion 330 to remove the disc material 630 positioned about the disc space of the surgical site 600.

In summary, the present disclosure generally relates to an expandable reamer having movable blades that may be positioned at an initial smaller diameter and expanded to a subsequent diameter to ream and/or drill a larger diameter at a surgical site. Such an expandable reamer may be useful for enlarging a borehole (or removing disc material 630) within the surgical site below a particular depth, since the expandable reamer may be disposed within a borehole (or surgical space) of an initial diameter and expanded, rotated, and displaced to form an enlarged borehole therebelow.

The expandable reamer of the present disclosure may be configured so that the movable blades expand to an outermost radial or lateral position under selected operating conditions, as well as return to an inward radial or lateral position under selected operating conditions. Furthermore, movable blades disposed within the expandable reamer of the present disclosure may comprise tapered, spiral, or substantially straight longitudinally extending sections extending from the tubular channel of the expandable reamer. It also may be advantageous to shape the movable blades so that the longitudinal sides of the movable blades are not straight. For instance, each longitudinal side of the movable blades may comprise an oval, elliptical, or other arcuate shape. Of course, the sides need not be symmetrical, but may be if so desired. Such a configuration may reduce binding of the movable blades as they move radially or laterally inwardly and/or outwardly.

Advantageously, the present disclosure provides a low-cost and potentially disposable reamer assembly that provides a predefined reamer body shape which is expandable after insertion into the bone structure, which includes deformable blades that are secured at distal ends thereof, and which may include a distal end cutter for boring the initial bore into the bone structure and/or removing disc material 630 between vertebral bodies.

The disc reamer of the described embodiments improves the quality of disc removal and endplate preparation while minimizing the trauma of surgery, minimizing blood loss, and markedly reducing surgical time.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

It will be understood that various modifications may be made to the embodiments of the presently disclosed expandable reamer assembly. Therefore, the above description should not be construed as limiting, but merely as exemplifications of embodiments. Those skilled in the art will envision other modifications within the scope and spirit of the present disclosure.

What is claimed is:

1. A surgical reamer assembly comprising:
a dilator having a longitudinal passage extending therethrough for receiving a guidewire;
a tubular channel positioned over the dilator; and
an expandable reamer head coupled to an elongated shaft, such that the expandable reamer head is insertable through the tubular channel once the dilator and guidewire are removed.

2. The surgical reamer assembly according to claim 1, wherein a proximal end of the shaft is coupled to a driving source configured to rotatably actuate the expandable reamer head.

3. The surgical reamer assembly according to claim 1, wherein the expandable reamer head is configured to be shaped as one of conical, spherical and cylindrical.

4. The surgical reamer assembly according to claim 1, wherein the tubular channel includes an irrigation port for (i) communicating a fluid to enter the tubular channel and (ii) moving loose disc material away from the expandable reamer head.

5. The surgical reamer assembly according to claim 1, wherein the tubular channel has a threaded distal end configured to be fixedly secured to endplates of one or more vertebral bodies.

6. The surgical reamer assembly according to claim 1, wherein the tubular channel is configured to be releasably secured to a handle portion such that a substantially “L-shape” configuration is formed.

7. The surgical reamer assembly according to claim 1, wherein the expandable reamer includes at least one cutting element.

8. The surgical reamer assembly according to claim 7, wherein the at least one cutting element is a pair of opposed cutting blades.

9. The surgical reamer assembly according to claim 8, wherein in an expanded state the pair of opposed cutting blades are exposed and in a retracted state the pair of opposed cutting blades are concealed within the expandable reamer head.

10. A method of removing disc material between vertebral bodies, the method comprising:
introducing a dilator having a guidewire extending therethrough into a disc space for separating muscle and tissue;
positioning a tube channel over the dilator;
removing the dilator and the guidewire;
inserting an expandable reamer head through the tube channel for accessing the disc space;
expanding at least one portion of the expandable reamer head to expose at least one cutting element; and
manipulating the expandable reamer head to remove the disc material positioned about the disc space.

11. The method according to claim 10, further including an irrigation port for (i) communicating a fluid to enter the tubular channel and (ii) moving loose disc material away from the expandable reamer head.

12. The method according to claim 10, further including providing the tubular channel with a threaded distal end configured to be fixedly secured to endplates of one or more vertebral bodies.

13. The method according to claim 10, further including releasably securing a handle portion to the tubular channel such that a substantially “L-shape” configuration is formed.

14. The method according to claim 10, further including providing the expandable reamer with at least one cutting element.

15. The method according to claim 14, wherein the at least one cutting element is a pair of opposed cutting blades.

16. The method according to claim 15, wherein in an expanded state the pair of opposed cutting blades are exposed and in a retracted state the pair of opposed cutting blades are concealed within the expandable reamer head.

17. The method according to claim 10, wherein the manipulating step pertains to both axial movement and rotational movement of the expandable reamer head.

18. The method according to claim 10, further comprising providing a plurality of reaming assemblies each including an expandable reamer head.

19. The plurality of reaming assemblies according to claim 18, wherein one reaming assembly of the plurality of reaming assemblies is placed on an ipsilateral side of a disc space and another reaming assembly of the plurality of reaming assemblies is placed on a contralateral side of the disc space.
